(19) United States
(12) Patent Application Publication
(10) Pub. No.: US 2023/0330112 A1
(43) Pub. Date: Oct. 19, 2023

(54) COMPOSITION AND TREATMENT FOR CANCER WITH MUTATIONS

(71) Applicant: BRIDGE BIOTHERAPEUTICS, INC., Seongnam-si (KR)

(72) Inventors: Sang Yoon LEE, Seoul (KR); Yong-Hee Lee, Flemington, NJ (US); Sujin Jung, Seoul (KR); Eun Young Park, Seoul (KR)

(73) Assignee: BRIDGE BIOTHERAPEUTICS, INC., Seongnam-si (KR)

(21) Appl. No.: 18/300,890

(22) Filed: Apr. 14, 2023

Related U.S. Application Data
(60) Provisional application No. 63/331,355, filed on Apr. 15, 2022.

(51) Int. Cl. A61K 31/63; A61K 45/06; A61P 35/00

(52) U.S. Cl. CPC A61K 31/63; A61K 45/06; A61P 35/00

(57) ABSTRACT

A pharmaceutical composition including an effective amount of an EGFR inhibitor and uses thereof are disclose. The pharmaceutical composition is in a form of fixed daily dose formulation. The EGFR inhibitor may be used in manufacturing a medicament for treating a subject with cancer having mutations or ameliorating symptoms of the cancer in the subject. The EGFR inhibitor may be used in a treatment method of cancer having mutations or ameliorating symptoms of the cancer in a subject.

### CROSS REFERENCE TO RELATED APPLICATIONS

This application claims benefit from U.S. Provisional Application No. 63/331,355 filed Apr. 15, 2022, of which the content is incorporated by reference herein in its entirety.

### FIELD

The present disclosure generally relates to a pharmaceutical composition comprising an EGFR inhibitor that shows a high inhibitory ability on EGFR mutations and treatment of cancers with mutations using the pharmaceutical composition.

### BACKGROUND ART

A traditional approach to fight cancer is chemotherapy using cytotoxic drugs. But these agents are often poorly selective for cancer cells, subsequently leading to damage to normal cells that divide rapidly. Other treatment options include radiation therapy, targeted therapy, immunotherapy, hormonal therapy, and surgery. The choice of therapy depends upon various factors including the cancer type, severity of the tumor, the stage of the disease and general state of the patients.

Targeted therapeutic agents are generally prepared to show efficacy in patients by targeting malfunctioning proteins that cancer cells characteristically have. There are targeted therapies for lung cancer, colorectal cancer, head and neck cancer, breast cancer, multiple myeloma, lymphoma, prostate cancer, melanoma and other cancers (www.cancer.gov/about-cancer/treatment/typeshargeted-therapieshargeted-therapies-fact-sheet).

Among them, EGFR (epidermal growth factor receptor, also known as ErbB-1 or HER1 in humans) inhibitors have achieved remarkable outcomes in the treatment for patients with EGFR mutant cancers. EGFR is a transmembrane protein that is a receptor for members of the epidermal growth factor family (EGF family) of extracellular protein ligands and is known to be abnormally activated in a number of epithelial cell tumors including non-small cell lung carcinoma (NSCLC), breast cancer, glioma, squamous cell carcinoma of head and neck, colorectal cancer, rectal adenocarcinoma, head and neck cancer, gastric cancer, and prostate cancer. The activation of the EGFR-tyrosine kinase causes persistent cell proliferation, invasion of the surrounding tissue, remote metastasis, and angiogenesis, and increases tumor cell survival.

First-, second- and third-generation epidermal growth factor receptor (EGFR) inhibitors have substantially improved the survival of patients with advanced, EGFR-mutant non-small cell lung cancer (NSCLC). For example, osimertinib, a third-generation, irreversible and mutant-specific inhibitor, has improved progression-free survival (PFS) in patients with EGFR-mutant NSCLC, in both heavily pre-treated and un-treated populations, and in adjuvant settings.

However, acquired resistance invariably emerges after treatment with osimertinib, leading to disease progression (1). The predominant resistance mechanism following osimertinib treatment is a tertiary point mutation at the C797 residue of EGFR, in which the cysteine within the ATP-binding site is substituted with serine and, therefore, prevents the formation of a covalent bond between the mutant EGFR and Osimertinib. This C797S mutation has been found to account for 10-26% of osimertinib-resistant cases in second-line and 7% in frontline settings (2, 3). Furthermore, the C797S mutation also confers cross-resistance to all other third-generation EGFR tyrosine kinase inhibitors (TKIs), such as rociletinib and narzartinib due to their similar binding mode, greatly limiting treatment options for these patients (4). The current National Comprehensive Cancer Network Guidelines recommend frontline treatment with osimertinib for patients bearing EGFR exon 19 deletion (19Del) or L858R substitution. However, after progression on osimertinib, there are no approved targeted therapy options available, and patients must turn to local therapies or systemic chemotherapies for treatment. Anecdotal cases of efficacy from combinations of third- and first-generation EGFR TKIs, or brigatinib plus cetuximab have been reported, but do not represent generally accepted practice.

Currently, the combination of amivantamab, a bi-specific antibody to EGFR and c-MET (mesenchymal—epithelial transition factor), and lazertinib, a third-generation EGFR TKI, is being investigated in multi-national phase 1 clinical trials (CHRYSALIS, NCT02609776; CHRYSALIS-2, NCT04077463), and promising early data have been presented (5). Other clinical trials exploring the benefit of immuno-oncology therapeutic strategies such as immune checkpoint inhibitors are ongoing (e.g., KEYNOTE 789, NCT03515837; CHECKMATE 722, NCT02864251), but data on their effectiveness and safety for treating NSCLC are not yet available.

For non-small cell lung cancer (NSCLC) patients with disease progression on osimertinib, there is an urgent need for a next-generation EGFR TKI that is active against C797S-containing mutations. Preclinical evaluation of BLU-945, a fourth-generation EGFR TKI, has shown antitumor activity in an in vivo model of NSCLC (6) and is currently under clinical investigation either as monotherapy or in combination with osimertinib (NCT04862780).

WO2018/230934 and counterpart U.S. Pat. No. 11,253,516 B2 disclose N2,N4-diphenylpyrimidine-2,4-diamine compounds showing a relatively weak EGFR activity inhibitory effect on wild-type EGFR, but a high inhibitory ability on EGFR mutation. The compounds also showed a high inhibitory activity on FMS-like tyrosine kinase 3 (FLT3) and FLT3 mutations. The entire contents of WO2018/230934 and U.S. Pat. No. 11,253,516 B2 are incorporated herein by reference.

The present inventors surprisingly found that N-(2-((5-chloro-2-((2-methoxy-4-(4-(4-methylpiperazin-1-yl)piperidin-1-yl)phenyl)amino)pyrimidin-4-yl)amino)phenyl)methanesulfonamide (Compound A) shows excellent inhibitory, suppressive, and therapeutic effects on non-small cell lung cancer (NSCLC) with mutations. Compound A is a novel oral, reversible, fourth-generation EGFR TKI. Compound A is a highly potent inhibitor of EGFR C797S mutants and has broad selectivity over wild-type EGFR.

### SUMMARY

One aspect of the present disclosure relates to a pharmaceutical composition comprising or consisting essentially of N-(2-((5-chloro-2-((2-methoxy-4-(4-(4-methylpiperazin-1-yl)piperidin-1-yl)phenyl)amino)pyrimidin-4-yl)amino)phenyl)methanesulfonamide (Compound A), a prodrug thereof, a hydrate thereof, a solvate thereof, an isomer thereof, an isotope labeled thereof, or pharmaceutically acceptable salts thereof, as active ingredients, for treating patients with cancer including mutation(s) or ameliorating symptoms of cancer with mutations. The types of mutation(s) include single mutation, double mutation, and triple mutation. The mutations include EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S. In some embodiments, the patient has developed or is expected to develop resistant to an EGFR-TKI treatment.

According to embodiments, the pharmaceutical composition may comprise or consists essentially of Compound A at a fixed daily dose of 40 mg to 600 mg. In some embodiments, the fixed daily dose may be in a range from about 60 mg to about 550 mg, about 70 mg to about 550 mg, about 80 mg to about 500 mg, about 80 mg to 600 mg. In embodiments, the fixed daily dose may be about 20 mg to about 120 mg, about 120 mg to about 200 mg, about 200 mg to about 300 mg, about 300 mg to about 500 mg, or about 500 mg to 600 mg. In certain embodiments, the fixed daily dose may be about 300 mg, about 310 mg, about 320 mg, about 330 mg, about 350 mg, about 360 mg, about 370 mg, about 380 mg, about 390 mg, about 400 mg, about 410 mg, about 420 mg, about 430 mg, about 450 mg, about 460 mg, about 470 mg, about 480 mg, about 490 mg, or about 500 mg.

According to embodiments, the mutation-containing cancers may have developed a tolerance to various cancer treatments such as radiation therapy, chemotherapy, cancer immunotherapy, symptomatic treatment, etc.

In some embodiments, the subject may have received or is under a chemotherapy employing, but not being limited to, gefitnib, afatinib, osimertinib, lazertinib, erlotinib, or a combination thereof.

In some embodiments, the cancer with mutations may be lung cancer with mutations. In certain embodiment, the cancer with mutations may be non-small cell lung cancer (NSCLC) with EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S mutations.

One aspect of the present disclosure is a fixed dose daily formulation for treating cancer with mutations, wherein the fixed daily dose formulation comprises or consists essentially of, as an active ingredient, N-(2-((5-chloro-2-((2-methoxy-4-(4-(4-methylpiperazin-1-yl)piperidin-1-yl)phenyl)amino)pyrimidin-4-yl)amino)phenyl)methanesulfonamide (Compound A), a prodrug thereof, a hydrate thereof, a solvate thereof, an isomer thereof, an isotope labeled thereof, or pharmaceutically acceptable salts thereof, in an amount of about 60 mg to about 550 mg, about 70 mg to about 550 mg, about 80 mg to about 500 mg, about 80 mg to 600 mg in terms of Compound A. In some embodiments, the fixed daily dose of Compound A may be about 20 mg to about 120 mg, about 120 mg to about 200 mg, about 200 mg to about 300 mg, about 300 mg to about 500 mg, or about 500 mg to 600 mg. The formulation may comprise a pharmaceutically acceptable carriers. The cancer with mutations may be lung cancer with mutations. In some embodiments, the cancer with mutations may be non-small cell lung cancer (NSCLC) with EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S mutations.

One aspect of the present disclosure is a fixed daily dose formulation for treating cancer with mutations or ameliorating symptoms of cancer with mutations, wherein the fixed dose daily formulation comprises or consists essentially of, as an active ingredient, N-(2-((5-chloro-2-((2-methoxy-4-(4-(4-methylpiperazin-1-yl)piperidin-1-yl)phenyl)amino)pyrimidin-4-yl)amino)phenyl)methanesulfonamide (Compound A), a prodrug thereof, a hydrate thereof, a solvate thereof, an isomer thereof, an isotope labeled thereof, or pharmaceutically acceptable salts thereof, in an amount of 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 11 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg,126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 g, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 g, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 g, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 g, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 g, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 g, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 g, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 g, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 g, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 g, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 g, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 g, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 g, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 g, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 g, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, or 600 mg, in terms of Compound A. The formulation may comprise a pharmaceutically acceptable carriers. The cancer with mutations may be lung cancer with mutations. In some embodiments, the cancer with mutations may be non-small cell lung cancer (NSCLC) with EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S mutations.

In some embodiments, the above-discussed fixed daily dose formulations or the above-discussed pharmaceutical compositions may be administered alone or in combination with another treatment such as known chemotherapy, radiation therapy, cancer immunotherapy, or symptomatic treatment. In embodiments, the chemotherapy may include, but is not limited to, osimertinib (AZD9291), nazartinib (EGF816), mavelertinib (PF-06747775), avitinib (AC0010), azertinib (YH25448, GNS-1480), naquotinib (ASP8273), olmutinib (HM61713), rociletinib (CO-1686), erlotinib, gefitinib, afatinib, dacomitinib, or a combination thereof. In some other embodiments, the another treatment is a therapeutic antibody, wherein the therapeutic antibody is cetuximab, panitumumab, nimotuzumab or necitumumab.

An embodiment of the present disclosure relates to uses of the fixed daily dose formulations discussed above, in treating a subject with cancer(s) including mutations or ameliorating symptoms of cancer with mutations. According to some embodiments, the fixed daily dose formulations may be administered alone or in combination with another treatment such as known chemotherapy, radiation therapy, cancer immunotherapy, or symptomatic treatment. In embodiments, the chemotherapy may include, but is not limited to, osimertinib (AZD9291), nazartinib (EGF816), mavelertinib (PF-06747775), avitinib (AC0010), azertinib (YH25448, GNS-1480), naquotinib (ASP8273), olmutinib (HM61713), rociletinib (CO-1686), erlotinib, gefitinib, afatinib, dacomitinib, or a combination thereof. In some other embodiments, the another treatment is a therapeutic antibody, wherein the therapeutic antibody is cetuximab. The cancer with mutations may be lung cancer with mutations. In some embodiments, the cancer with mutations may be non-small cell lung cancer (NSCLC) with EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S mutations.

An embodiment of the present disclosure relates to uses of Compound A, a prodrug thereof, a hydrate thereof, a solvate thereof, an isomer thereof, an isotope labeled thereof, or pharmaceutically acceptable salts thereof, in manufacturing a medicine for treating a patient with cancer(s) including mutations. According to some embodiments, the medicine may be administered alone or in combination with another treatment such as known chemotherapy, radiation therapy, cancer immunotherapy, or symptomatic treatment. In embodiments, the chemotherapy may include, but is not limited to, osimertinib (AZD9291), nazartinib (EGF816), mavelertinib (PF-06747775), avitinib (AC0010), azertinib (YH25448, GNS-1480), naquotinib (ASP8273), olmutinib (HM61713), rociletinib (CO-1686), erlotinib, gefitinib, afatinib, dacomitinib, or a combination thereof. The another treatment can be a therapeutic antibody, wherein the therapeutic antibody is cetuximab, panitumumab, nimotuzumab or necitumumab. The cancer with mutations may be lung cancer with mutations. In some embodiments, the cancer with mutations may be non-small cell lung cancer (NSCLC) with EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S mutations.

An embodiment of the present disclosure relates to a method of treating a subject with cancer including mutations or ameliorating symptoms of cancer with mutations in a subject, comprising administering to the subject a therapeutically effective amount of a pharmaceutical composition comprising or consisting essentially of, as an active ingredient, N-(2-((5-chloro-2-((2-methoxy-4-(4-(4-methylpiperazin-1-yl)piperidin-1-yl)phenyl)amino)pyrimidin-4-yl)amino)phenyl)methanesulfonamide (Compound A), a prodrug thereof, a hydrate thereof, a solvate thereof, an isomer thereof, an isotope labeled thereof, or pharmaceutically acceptable salts thereof, in an amount from 20 mg to 600 mg daily. In certain embodiments, the therapeutically effective amount per day is a fixed daily dose of 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 11 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg,126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 g, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 g, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 g, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 g, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 g, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 g, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 g, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 g, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 g, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 g, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 g, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 g, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 g, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 g, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 g, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, or 600 mg, in terms of Compound A. The composition may comprise a pharmaceutically acceptable carriers. In some embodiments, the pharmaceutical composition may be a daily fixed dose formulation. The cancer with mutations may be lung cancer with mutations. In some embodiments, the cancer with mutations may be non-small cell lung cancer (NSCLC) with EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S mutations. According to some embodiments, the pharmaceutical composition may be administered alone or in combination with known chemotherapy, radiation therapy, cancer immunotherapy, or symptomatic treatment. In embodiments, the chemotherapy may include, but is not limited to, osimertinib (AZD9291), nazartinib (EGF816), mavelertinib (PF-06747775), avitinib (AC0010), azertinib (YH25448, GNS-1480), naquotinib (ASP8273), olmutinib (HM61713), rociletinib (CO-1686), erlotinib, gefitinib, afatinib, dacomitinib, or a combination thereof. The another treatment can be a therapeutic antibody, wherein the therapeutic antibody is cetuximab, panitumumab, nimotuzumab or necitumumab. The cancer with mutations may be lung cancer with mutations. In some embodiments, the cancer with mutations may be non-small cell lung cancer (NSCLC) with EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S mutations.

According to still another aspect, disclosed is a method for treating cancer in a subject in need thereof, the method comprising: a) identifying a subject with cancer having an EGFR gene mutation; and b) administering to the identified subject an effective amount of Compound A, a prodrug thereof, a hydrate thereof, a solvate thereof, an isomer thereof, an isotope labeled thereof, or a pharmaceutically acceptable salt thereof, wherein the effective amount is between 20 mg and 600 mg of Compound A. In embodiments, the EGFR gene mutation comprises L858R, L858R/T790M, L858R/C797S, L858R/T790M/C797S, Del19, Del19/T790M, Del19/C797S, or Del19/T790M/C797S. In embodiments, the cancer is a lung cancer. In certain embodiment, the cancer is a non-small cell lung cancer.

In the above-discussed pharmaceutical compositions, the daily fixed dose formulations, the uses in the treatment of cancer, the uses in the manufacturing a cancer medicine, and the treatment methods, the compositions or formulations may be administered at daily (once or twice or three times a day), every other day, or an interval of 5 days to 1 month, 7 days to 1 month, 1 week to 4 weeks, 2 weeks to 4 weeks, 2 weeks to 3 weeks, 1 week, 10 days, 2 weeks, 3 weeks, 4 weeks, or twice a month.

### BRIEF DESCRIPTION OF THE DRAWINGS

So that the manner in which the above recited features of the present disclosure can be understood in detail, a more particular description of the disclosure, briefly summarized above, may be had by reference to the embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 shows the AUC (area under curve) of Compound A at doses of 20 mg, 80 mg, 160 mg, 320 mg, and 480 mg, respective, at day 1 of treatment cycle 1 and the treatment cycle 2.

FIGS. 2A-2D show the combination effects of Compound A plus osimeritinib or cetuximab against EGFR mutations.

FIGS. 3A and 3B show the suppressive effects of EGFR-mutant lung cancer by Compound A in vivo.

FIGS. 4A and 4B are computed tomography (CT) scan images of two patients reported in Experimental Example 2, Clinical Study, Case 1 and Case 2, respectively.

FIG. 5 is the duration of treatment and tumor response in all patients in QD dosing cohorts by dose level. *: In four patients, EGFR mutations were not detected from repeated assessment of ctDNA. Abbreviations in FIG. 5 have the following meaning: ctDNA, circulating tumor DNA; G, glycine; ND, not detected; QD, once daily; SAE, serious adverse event.

FIG. 6 illustrates the protocol for human clinical study described in Experimental Example 2, Clinical Study.

### DETAILED DESCRIPTION

### Definitions

The general terms used herein are defined with the following meanings, unless explicitly stated otherwise:

The terms “a” and “an” and “the” and similar references in the context of describing the disclosure (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “about” or “approximately” shall have the meaning of within 10%, more preferably within 5%, of a given value or range.

The term “IC₅₀” refers to an amount, concentration or dosage of a particular test compound that achieves a 50% inhibition of a maximal response, such as cell growth or proliferation, measured via any of the in vitro or cell based assay described herein.

The terms “comprising” and “including” are used herein in their open-ended and non-limiting sense unless otherwise noted. The terms “consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

The present disclosure embodiments also include pharmaceutically acceptable salts of the compounds useful according to the disclosure described herein. As used herein, “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. Suitable organic acids are, e.g., carboxylic acids or sulfonic acids, such as acetic acid, succinic acid, fumaric acid or methansulfonic acid. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. For example, the salt is a sulphate salt, or bisulphate salt. In another embodiment, the salt is a succinic salt.

The term “prodrug” refers to an inactive precursor of an agent that is converted into a biologically active form in vivo. Prodrugs are often useful because, in some situations, they may be easier to administer than the parent compound. The prodrug may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977).

The term “hydrate” means a compound provided herein or a salt thereof, that further includes a stoichiometric or non-stoichiometric amount of water bound by non-covalent intermolecular forces.

The term “solvate” means a solvate formed from the association of one or more solvent molecules to a compound provided herein. The term “solvate” includes hydrates (e.g., mono-hydrate, dihydrate, trihydrate, tetrahydrate and the like).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “administration” refers to introducing an agent of the present disclosure into a subject or patient. One preferred route of administration of the agents is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

The terms “co-administration” or “combined administration” or “combined use” or “in combination with” are meant to encompass administration of two therapeutic agents (for example, Compound A provided herein and another anti-cancer agent) to a single subject in need thereof (e.g. a patient), and are intended to include treatment regimens in which the two agents are administered simultaneously, concurrently, or sequentially with no specific time limits. In embodiments, both agents are present in the cell or in the patient's body at the same time or exert their biological or therapeutic effect at the same time. In embodiments, the two therapeutic agents are in the same composition or unit dosage form. In another embodiments, the two therapeutic agents are in separate compositions or unit dosage forms.

The term “anti-cancer agents” refers to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxorubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent® and Bevacizumab) or any other cytotoxic agents, (estramustine phosphate, prednimustine), hormones or hormone agonists, antagonists, partial agonists or partial antagonists, kinase inhibitors, radiation treatment, or therapeutic antibody.

The term “pharmaceutical composition” or “formulation” is defined herein to refer to a mixture or solution containing at least one therapeutic agent to be administered to a subject, e.g., a mammal or human, in order to prevent or treat a particular disease or condition affecting the subject or patient including mammal or human.

The term “pharmaceutically acceptable excipient” or “pharmaceutically acceptable carrier” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use.

The term “unit dosage form,” as used herein, refers to physically discrete units suitable as unitary dosages for human and/or animal subjects, each unit containing a predetermined quantity of a compound (e.g., Compound A, a prodrug thereof, an isomer thereof, a solvate thereof, a hydrate thereof, or a pharmaceutically acceptable thereof as described herein) calculated in an amount sufficient to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the subject or patient.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease, and/or suppressing the increase in tumor volume or size, or reducing the tumor volume or size.

The terms “ameliorate,” “ameliorating” and grammatical variations thereof mean to decrease the severity of the symptoms of a disease in a subject.

The term “protect” is used herein to mean prevent delay or treat, or all, as appropriate, development or continuance or aggravation of a disease in a subject, e.g., a mammal or human.

The term “prevent”, “preventing” or “prevention” as used herein comprises the prevention of at least one symptom associated with or caused by the state, disease or disorder being prevented.

The term “effective amount” or a “therapeutically effective amount” of Compound A or pharmaceutical compositions containing Compound A is an amount of Compound A or composition that is sufficient to effect beneficial or desired results when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of an agent or composition will be that amount of the agent or composition, which is the lowest dose effective to produce the desired effect. The effective dose of an agent or composition may be administered as two, three, four, five, six or more sub-doses, administered separately at appropriate intervals throughout the day.

The term “subject” or “patient” as used herein includes animals, which are capable of suffering from or afflicted with a cancer or any disorder involving, directly or indirectly, a cancer. Examples of subjects include mammals, e.g., humans, dogs, cows, horses, pigs, sheep, goats, cats, mice, rabbits, rats and transgenic non-human animals. In the preferred embodiment, the subject is a human, e.g., a human suffering from, at risk of suffering from, or potentially capable of suffering from cancers.

### Compound A

According to the present disclosure, Compound A, i.e., N-(2-((5-chloro-2-((2-methoxy-4-(4-(4-methylpiperazin-1-yl)piperidin-1-yl)phenyl)amino)pyrimidin-4-yl)amino)phenyl)methanesulfonamide (Compound A) has he following structure:

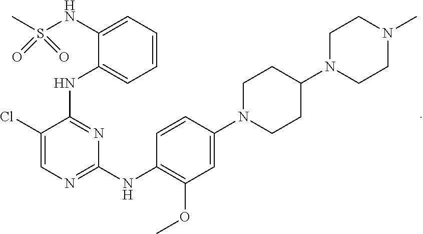

A prodrug, a hydrate, a solvate, an isomer, an isotope labeled compound, or a pharmaceutically acceptable salt of Compound A is also encompassed within the scope of the present disclosure. The doses or the effective amounts as used herein is calculated as amount of Compound A.

### Pharmaceutical Composition, Formulations, and Uses

An aspect of the present disclosure also pertains to a pharmaceutical composition or formulation or medicine comprising or consisting essentially of, as an active ingredient, an effective amount of Compound A, a prodrug thereof, a hydrate thereof, a solvate thereof, an isomer thereof, an isotope labeled compound thereof, or a pharmaceutically acceptable salt thereof, wherein the effective amount is a daily dosage between 20 mg and 600 mg. The composition or formulation or medicine may further comprise pharmaceutically acceptable carriers.

The inventors surprisingly found that Compound A does not show adverse effects in early human study at a fixed daily dose of about 600 mg. The pharmaceutical composition or formulation or medicine may be administered at a fixed daily dose of 40 mg to 600 mg. In some embodiments, the dose may be in a range from about 60 mg to about 550 mg, about 70 mg to about 550 mg, about 80 mg to about 500 mg, about 80 mg to 600 mg of Compound A. In some embodiments, the fixed dose is about 20 mg to about 120 mg, about 120 mg to about 200 mg, about 200 mg to about 300 mg, about 300 mg to about 500 mg, or about 500 mg to 600 mg, per day.

In some embodiment, the fixed daily dose may be about 300 mg, about 310 mg, about 320 mg, about 330 mg, about 350 mg, about 360 mg, about 370 mg, about 380 mg, about 390 mg, about 400 mg, about 410 mg, about 420 mg, about 430 mg, about 450 mg, about 460 mg, about 470 mg, about 480 mg, about 490 mg, or about 500 mg.

A fixed daily dose of Compound A may be 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 11 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg,126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 g, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 g, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 g, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 g, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 g, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 g, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 g, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 g, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 g, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 g, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 g, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 g, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 g, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 g, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 g, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, or 600 mg.

A pharmaceutical composition or formulations or medicines comprising or consisting essentially of Compound A may be administered at daily (once or twice or three times a day), every other day, or an interval of 5 days to 1 month, 7 days to 1 month, 1 week to 4 weeks, 2 weeks to 4 weeks, 2 weeks to 3 weeks, 1 week, 10 days, 2 weeks, 3 weeks, 4 weeks, or twice a month. A particular dosage and its administration cycle can be determined and regulated by each patient's health condition, age, weight, tumor response to the drug, and the like.

Human clinical study results shows that the tumor volume was significantly reduced in non-small cell lung cancer (NSCLC) patients with a single mutation, double mutation, or triple mutation.

The composition or the fixed daily dose or unit dosage formulation according to the aspects, optionally comprising or being administered in combination with another chemotherapeutic agent, can be used for the treatment of proliferation disease or cancer with mutations. The nature of proliferative diseases is multifactorial, and drugs with different mechanisms of action may be combined.

For example, the pharmaceutical composition may be administered alone or in combination with known chemotherapy, radiation therapy, cancer immunotherapy, or symptomatic treatment. In embodiments, the chemotherapy may include, but is not limited to, osimertinib (AZD9291), nazartinib (EGF816), mavelertinib (PF-06747775), avitinib (AC0010), azertinib (YH25448, GNS-1480), naquotinib (ASP8273), olmutinib (HM61713), rociletinib (CO-1686), erlotinib, gefitinib, afatinib, dacomitinib, or a combination thereof. The another treatment can be a therapeutic antibody, wherein the therapeutic antibody is cetuximab, panitumumab, nimotuzumab or necitumumab.

In the disclosure, the combination composition can be used to treat proliferative diseases. The proliferative diseases can be a cancer that is associated with one, two, three, or more mutations in EGFR that activates EGFR or causes resistance to EGFR. In embodiments, the EGFR mutations may include del19, L858R, T790M, C797S, or a combination thereof. In an embodiment, the EGFR mutations may be del19, del19/L858R, del19/T790M, del19/C797S, L858/T790M, L858R/C797S, T790M/C797S, del19/T790M/C797S (DTC) or L858R/T790M/C797S (LTC).

In another aspect, cancer includes pseudomyxoma, intrahepatic cholangiocarcinoma, hepatoblastoma, liver cancer, thyroid cancer, colon cancer, testis cancer, myelodysplastic syndrome, glioblastoma, oral cancer, lip cancer, mycosis fungoides, acute myeloid leukemia, acute lymphocytic leukemia, basal cell carcinoma, epithelial ovarian cancer, ovarian seminoma, male breast cancer, brain cancer, pituitary adenoma, multiple myeloma, gallbladder cancer, cholangiocarcinoma, colorectal cancer, chronic myeloid leukemia, chronic lymphocytic leukemia, retinoblastoma, choroidal melanoma, ampullar of vater cancer, bladder cancer, peritoneal cancer, parathyroid cancer, adrenal cancer, nasal and paranasal cavity cancer, non-small cell lung cancer, tongue cancer, astrocytoma, small cell lung cancer, pediatric brain cancer, pediatric lymphoma, pediatric leukemia, small intestine cancer, meningioma, esophageal cancer, glioma, renal pelvis cancer, renal cancer, heart cancer, duodenal cancer, malignant soft tissue cancer, malignant bone cancer, malignant lymphoma, malignant mesothelioma, malignant melanoma, eye cancer, vulvar cancer, ureteral cancer, urethral cancer, cancer of unknown primary site, gastric lymphoma, gastric cancer, gastric carcinoid, gastrointestinal stromal tumor, Wilms' tumor, breast cancer, sarcoma, penile cancer, pharyngeal cancer, gestational choriocarcinoma, cervical cancer, endometrial cancer, uterine sarcoma, prostate cancer, metastatic bone cancer, metastatic brain cancer, mediastinal cancer, rectal cancer, rectal carcinoid, vaginal cancer, spinal cancer, vestibular schwannoma, pancreatic cancer, salivary gland cancer, Kaposi's sarcoma, Paget's disease, tonsillar cancer, squamous cell cancer, adenocarcinoma of lung, lung cancer, squamous cell lung cancer, skin cancer, anal cancer, rhabdomyosarcoma, laryngeal cancer, pleura cancer, and thymic cancer; and more preferably, the cancer may be a cancer with mutation on one or more selected from the group consisting of EGFR. In certain embodiment, the cancer is lung cancer. In another certain embodiments, the cancer is non-small cell lung cancer with mutations such as del19, del19/L858R, del19/T790M, del19/C797S, L858/T790M, L858R/C797S, T790M/C797S, del19/T790M/C797S (DTC), or L858R/T790M/C797S (LTC).

Suitable pharmaceutical compositions or formulations include for example tablets, capsules, suppositories, solutions, particularly solutions for injection (s.c., i.v., i.m.) and infusion, syrups, elixirs, emulsions or dispersible powders.

The pharmaceutical composition or formulation can be either administered in a single formulation or unit dosage form, administered concurrently, but optionally separately, or administered sequentially by any suitable route. The unit dosage form may also be a fixed daily dose formulation.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatin, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules comprising one or more active substances or combinations of active substances may for example be prepared by mixing the active substances with inert carriers such as lactose or sorbitol and packing them into gelatin capsules.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The compositions are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatin and the like. Moreover, lubricants such as magnesium stearate, sodium lauryl sulphate and talc may be used at the same time for the tabletting process. In the case of aqueous suspensions the active substances may be combined with various flavor enhancers or colorings in addition to the excipients mentioned above.

For parenteral use, solutions of the active ingredients with suitable liquid carriers may be used.

Before describing several exemplary embodiments of the disclosure, it is to be understood that the disclosure is not limited to the details of construction or process steps set forth in the following description. The disclosure is capable of other embodiments and of being practiced or being carried out in various ways.

### EXPERIMENTAL EXAMPLE 1: PRE-CLINICAL STUDY

### 1-1. Materials and Methods

The murine pro-B cell line Ba/F3 (RCB0805) and myelomonocytic, macrophage-like, BALB/C mouse leukemia cells (WEHI-3) were provided by the RIKEN Bio Resource Center (Tsukuba, Japan). Ba/F3 cells were maintained in RPMI 1640 medium (supplemented with 10% fetal bovine serum, 1% penicillin-streptomycin and conditioned media from WEHI-3 (10%) as a source of IL-3, and cultured at 37° C. in a humid atmosphere with 5% CO₂.

Ba/F3 cells with EGFR mutations were treated with Compound A with or without cetuximab at the indicated concentrations for 6 hours. The cells were then washed twice with PBS and resuspended in lysis buffer. Lysates were quantified using a BCA protein assay (Bio-Rad), and were electrophoresed and transferred to polyvinylidene difluoride membranes. Immunoblotting was performed using an antibody (Cell Signaling Technology, Danvers, MA, USA) according to the antibody manufacturers' instructions and were scanned using an AMERSHAM™ Imager 680.

### 1-2. Establishment of Ba/F3 Cells Expressing Mutant EGFRs

A pBABE-puro retroviral vector with human EGFR exon 19 deletion (E746_A750 del), exon deletion 19 plus T790M, V769insASV and H773insH were purchased from ADDGENE (Cambridge, MA), respectively. Using these as templates, the intended EGFR mutations were generated by the PRIME STAR™ Mutagenesis Basal Kit (TAKARA BIO). To generate the viral envelope, the pBABE-puro retroviral vector carrying each EGFR mutation was co-transfected with a pVSV-G vector (CLONTECH) to gpIRES-293 cells using the FuGENE6 transfection reagent (PROMEGA). After 48 hours of transfection, the supernatant of culture medium was collected, and the viral particles were concentrated by centrifugation and added to Ba/F3 cells.

### 1-3. Establishment of Compound A-Resistant Clones Through ENU Mutagenesis

Ba/F3 cells expressing EGFR mutations were exposed to 100 m/mL ENU for 24 hours. The cells were washed and cultured in RPMI with 10% FBS for 24 hours. Subsequently, 1×104 cells were plated in 96-well plates in the presence of Compound A. The medium containing Compound A was changed twice weekly for 3 weeks. When the cell growth was observed macroscopically, the cells were transferred to 48-well plates and incubated until they reached confluence in the absence of drug. Using the RNeasy Plus Mini Kit (QIAGEN), total RNA was isolated from resistant clones. Then, RNA was converted to cDNA using ReverTra Ace (TOYOBO) and total EGFR cDNAs were sequenced using a Genetic Analyzer 3130 or 3500XL (APPLIED BIOSYSTEMS).

### 1-4. Cell Growth-Inhibition Assay

A total of 2×10³ cells were seeded in each well of 96-well plates. After 24 hours, EGFR TKIs were added at the indicated concentrations. After a 72-hour incubation, 10 μL of the Cell Counting Kit-8 solution (DOJINDO LABORATORIES) was added to each well, and the plates were incubated for an additional 3 hours. Absorbance at 450 nm was measured using a multiplate reader (Tecan). The percentage of viable cells was evaluated and compared with those of DMSO-treated controls or PBS-treated controls.

### 1-5. Xenograft Studies

For Ba/F3 EGFR 19Del/C797S and EGFR 19Del/T790M/C797S xenografts models, each mouse was inoculated subcutaneously at the right flank with cells (0.5×10⁶) in 0.1 mL of PBS supplemented with BD Matrigel (1:1) for tumor development. The animals were randomized, and treatment was started when the average tumor volume reached 150-180 mm³ for the efficacy study.

For PDX models, fresh tumor tissues from mice bearing established primary cancer tissues were harvested and cut into small pieces (˜2-3 mm in diameter). Tumor fragments were inoculated subcutaneously at the upper right dorsal flank into corresponding female BALB/c nude mice or female nu/nu mice, ages 35-42 days, for tumor development. The randomization started when the mean tumor size reached approximately 150-200 mm³. Implanted mice were treated with vehicle or Compound A (60 and 90 mg/kg, N=8), once daily by oral gavage for the indicated period.

Tumor volumes were measured twice weekly in two dimensions using a caliper, and the volume was expressed in mm³ using the formula: volume=0.5 a×b² where a and b are the long and short diameters of the tumor, respectively. The tumor sizes were then used for the calculations of T/C (treatment/control) values. The tumor volume was used for calculation of tumor growth index (TGI) of each group according to the following formula: TGI (%)=[1-(Tₜᵢ-Tₜ₀)/(V_{ci}-V_{c0})]×100. The bodyweight of mice was measured daily for the dosing phase, and the relative change of bodyweight (RCBW) of each mouse according to the following formula:

RCBW (%)=(BWᵢ−BW₀)/BW₀×100.

### 1-6. Pharmacodynamic Analysis

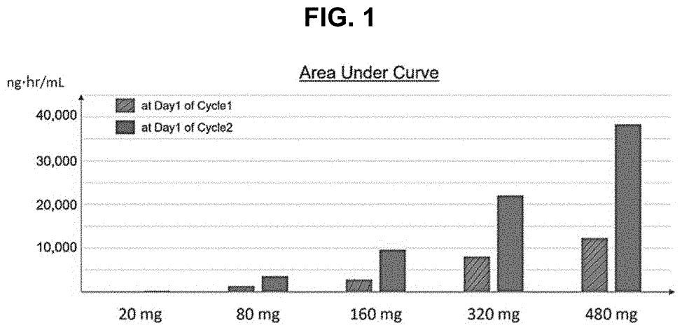

After the end of the patient-derived EGFR 19Del/T790M/C797S xenograft study, tumor tissue samples were collected from the mice and cut to about 30-100 mg and then were placed in a 2 mL microcentrifuge tube, adding the protein extraction buffer (Cell Signaling Technology) with protease inhibitors, and grinding the tumors with Tissuelyser (Qiagen) at 50 Hz for 5 min. The same amount of protein (30 μg) was then obtained from each suspension and subjected to 10% SDS-PAGE after which the separated proteins were transferred to a nitrocellulose membrane. After blocking with buffer containing 2.5% skim milk, the membrane was incubated overnight with primary antibodies. Blots were then washed and incubated with horseradish peroxidase (HRP)-anti-rabbit or HRP-anti-mouse antibodies. The AUC results are shown in FIG. 1.

### 1-7. The Combination Effects

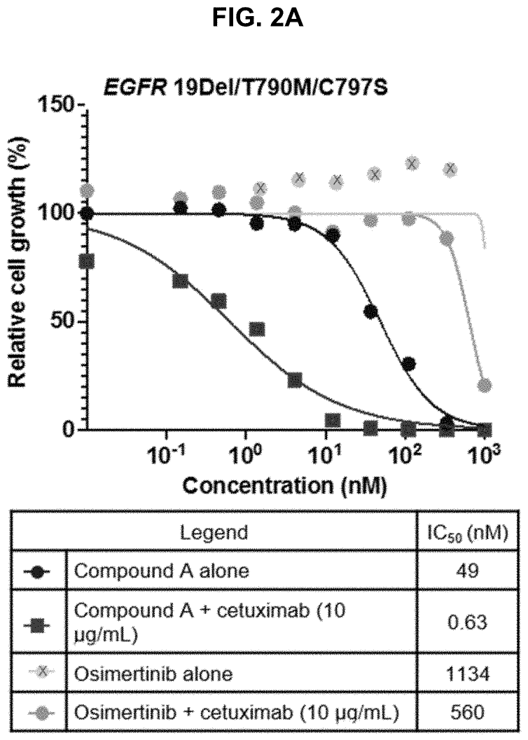

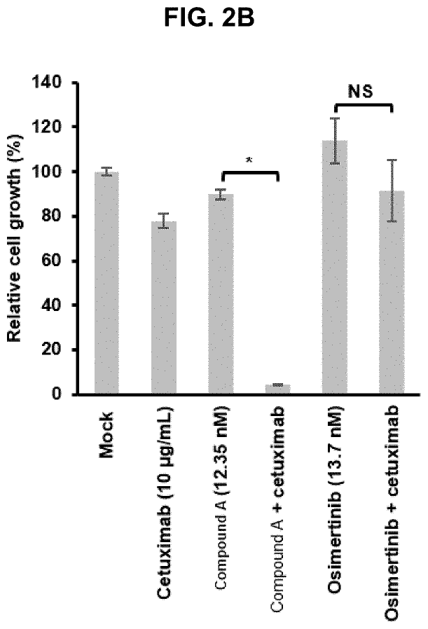

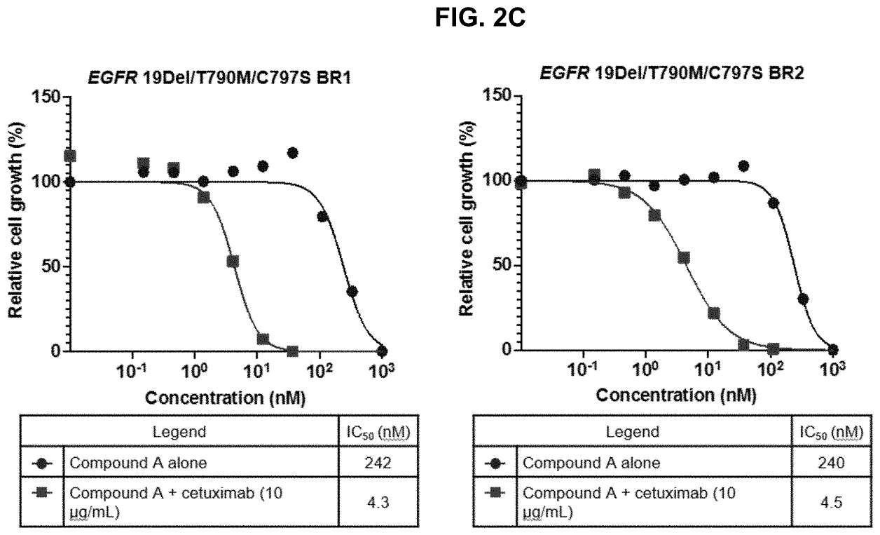

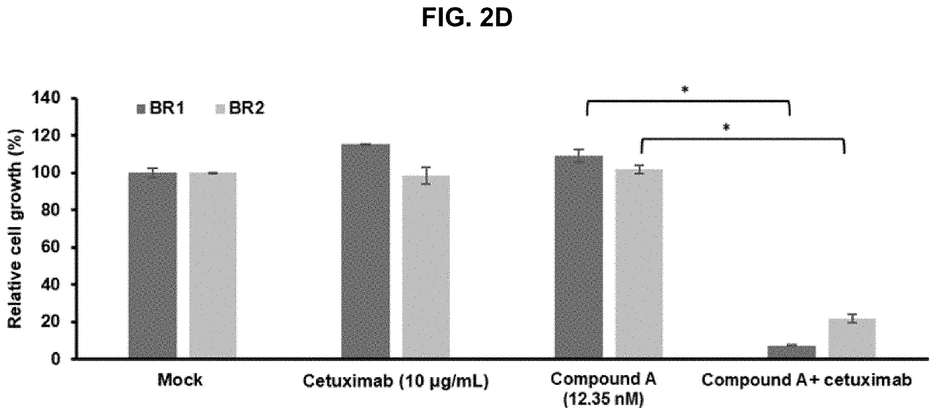

The combination effect of osimertinib and cetuximab against 19Del/T790M/C797S was evaluated, but the decrease in IC50 values was only about half (FIG. 2A). Compound A combined with cetuximab synergistically suppressed the growth of EGFR 19Del/T790M/C797S-expressing cells, whereas no synergistic benefit was obtained when combined with osimertinib (FIG. 2B). Furthermore, we tested the combination of Compound A and cetuximab against the resistant clones obtained by the ENU mutagenesis. Cetuximab alone was able to overcome Ba/F3 cells carrying EGFR 19Del or 19Del/C797S-derived Compound A-resistant clones as well as in parental cells. In Compound A-resistant Ba/F3 cells expressing EGFR 19Del/T790M/C797S, the enhanced activity of Compound A combined with cetuximab was observed, with IC50 lower than 5 nM, and a synergistic suppression of cancer cell growth (FIGS. 2C and 2D).

### 1-8. Suppression of EGFR-Mutant Lung Cancer by Compound A Cancer in vivo

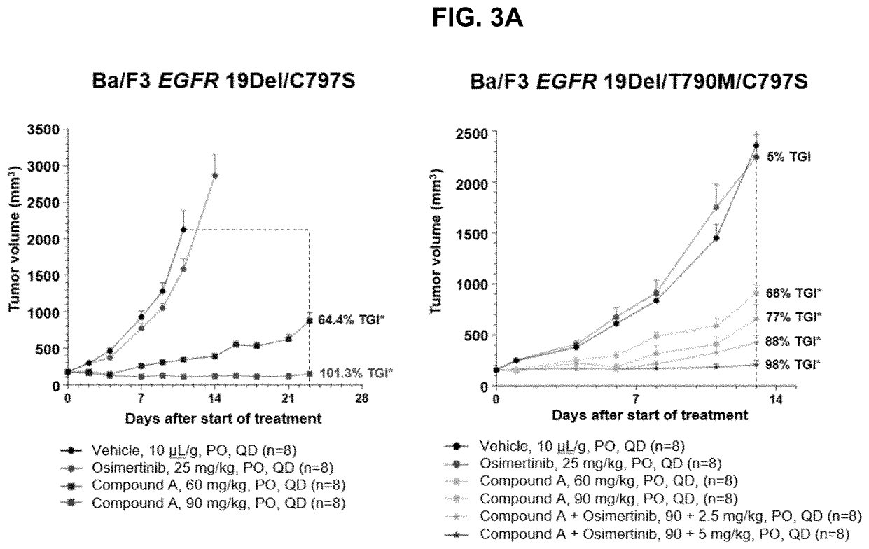

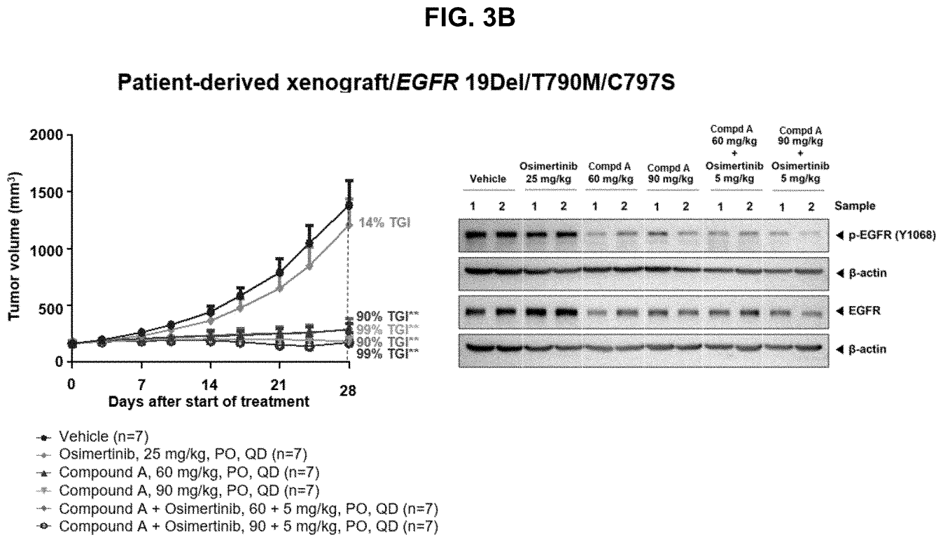

To determine whether Compound A is effective in animal models, we tested Compound A in mice bearing Ba/F3 EGFR 19Del/C797S or Ba/F3 EGFR 19Del/T790M/C797S xenografts. As shown in FIG. 3A, Compound A suppressed tumor growth in a dose-dependent manner with complete inhibition at 90 mg/kg in the Ba/F3 EGFR 19Del/C797S xenograft. In combination with osimertinib, Compound A at 90 mg/kg also resulted in complete tumor growth inhibition in Ba/F3 EGFR 19Del/T790M/C797S xenografts (FIG. 3A). In the vehicle-treated groups, both Ba/F3 EGFR 19Del/C797S and Ba/F3 EGFR 19Del/T790M/C797S xenografts grew continuously, and osimertinib did not block the tumor growth even at 25 mg/kg, which indicates osimertinib was not active for EGFR 19Del/C797S and EGFR 19Del/T790M/C797S.

The anti-tumor efficacy of Compound A against EGFR 19Del/T790M/C797S was also confirmed in a LD1-0025-200717 EGFR 19Del/T790M/C797S PDX model. The LD1-0025-200717 PDX model was derived from a patient who went through seven lines of therapy, including chemotherapy, erlotinib and osimertinib. As shown in FIG. 3B (left), once-daily dosing of Compound A induced significant tumor growth inhibition, with complete tumor growth inhibition observed at dose of 90 mg/kg/day and only a small decline in bodyweight. The tumor regression with only a small decline in bodyweight was also observed after the administration of 60 mg/kg of Compound A in a LU1235 EGFR 19Del PDX model (derived from a patient with poorly differentiated adenocarcinoma) for 21 days. As shown in FIG. 3B (right), 66.5-77.5% of inhibition of p-EGFR was observed in tumor samples from 90 mg/kg of Compound A or 90 mg/kg of Compound A and 5 mg/kg of osimertinib combination groups. These results clearly indicate Compound A has potent therapeutic efficacy against EGFR 19Del/C797S, EGFR 19Del/T790M/C797S and EGFR 19Del disease models.

### EXPERIMENTAL EXAMPLE 2: CLINICAL STUDY

### 2-1. Phase 1 Study of Compound A for NSCLC

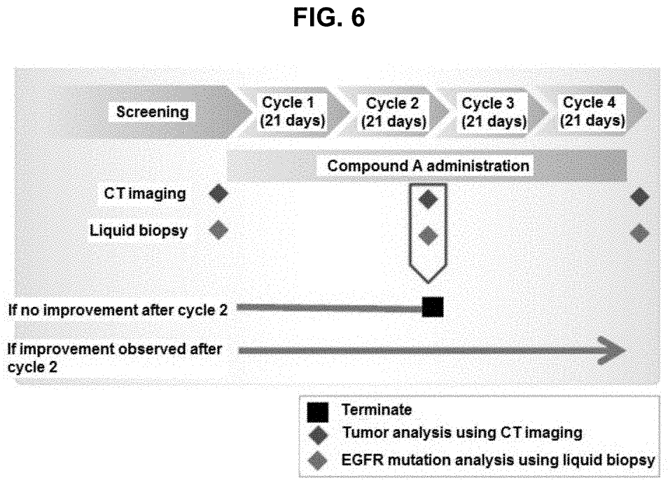

A first-in-human phase 1 study is ongoing to evaluate the safety of Compound A, including a dose-escalation phase to characterize dose-limiting toxicities (DLTs) (FIG. 6) and determine the recommended phase 2 dose (NCT04820023). Eligibility criteria included advanced-stage NSCLC with an activating EGFR mutation and disease progression on at least one prior EGFR inhibitor. A measurable lesion was required for exploratory evaluation of efficacy. Starting from 20 mg QD (once daily; every day), Compound A dose levels were escalated after evaluating dose-DLT relationships and toxicity probabilities during the first 21 days of dosing. Table 1 summarizes the phase 1 study population.

TABLE 1

| | All patients | |
|---|---|---|
| Characteristic | (N = 25), n (%) | |
| Median age (range) | 63 | (38-79) |
| Female | 17 | (68%) |
| Asian | 25 | (100%) |
| ECOG PS (0, 1) | 6 (24%), 19 (76%) | |
| Number of prior systemic anticancer regimens | | |
| 1 | 2 | (8%) |
| 2 | 7 | (28%) |
| ≥3 | 16 | (64%) |
| Prior EGFR TKI treatment | 25 | (100%) |
| Prior gefitinib, erlotinib, afatinib or dacomitinib | 25 | (100%) |
| Prior osimertinib or lazertinib | 20 | (80%) |
| Brain metastasis, stable (%) | 10 | (40%) |
| EGFR mutation detected by ctDNA (19Del, L858R) | 13 (52%), 8 (32%)* | |
| containing C797S | 8 | (32%) |

All enrolled patients underwent ctDNA analysis (Guardant360, Guardant Health) at screening and every 6 weeks throughout treatment, which was compared to imaging at the same time points. Changes in allelic frequency were calculated as per the proprietary algorithm of Guardant Health (7). Intra-patient dose-escalation to the next dose level was allowed. Plasma samples were collected at prespecified time points at cycle 2, Day 1 following multiple doses (steady state) of Compound A in the dose-escalation cohorts, and the plasma concentrations were determined by a validated LC-MS/MS method with the LLOQ established at 1 ng/mL.

### 2-2. Results

Tables 2 and 3 summarize the phase 1 study safety and treatment response to Compound A.

TABLE 2

| | 20 mg QD (N = 4) | | | | 80 mg QD (N = 3) | | | | 160 mg QD (N = 4) | | | |
|---|---|---|---|---|---|---|---|---|---|---|---|---|
| | All grade | | Grade ≥ 3 | | All grade | | Grade ≥ 3 | | All grade | | Grade ≥ 3 | |
| | N | % | N | % | N | % | N | % | N | % | N | % |
| Any TEAE* | 3 | 75% | 1 | 25% | 2 | 67% | 0 | 0% | 3 | 75% | 0 | 0% |
| TEAE occurring in ≥10% | | | | | | | | | | | | |
| of all patients | | | | | | | | | | | | |
| Nausea | 0 | 0% | 0 | 0% | 0 | 0% | 0 | 0% | 1 | 25% | 0 | 0% |
| Vomiting | 0 | 0% | 0 | 0% | 1 | 33% | 0 | 0% | 1 | 25% | 0 | 0% |
| Diarrhea | 0 | 0% | 0 | 0% | 0 | 0% | 0 | 0% | 0 | 0% | 0 | 0% |
| Anorexia | 0 | 0% | 0 | 0% | 0 | 0% | 0 | 0% | 0 | 0% | 0 | 0% |
| Constipation | 0 | 0% | 0 | 0% | 1 | 33% | 0 | 0% | 1 | 25% | 0 | 0% |
| Dizziness | 0 | 0% | 0 | 0% | 0 | 0% | 0 | 0% | 0 | 0% | 0 | 0% |
| Dyspepsia | 0 | 0% | 0 | 0% | 1 | 33% | 0 | 0% | 0 | 0% | 0 | 0% |
| Platelet count | 0 | 0% | 0 | 0% | 0 | 0% | 0 | 0% | 0 | 0% | 0 | 0% |
| decreased | | | | | | | | | | | | |
| Neutropenia/Neutrophil | 0 | 0% | 0 | 0% | 0 | 0% | 0 | 0% | 0 | 0% | 0 | 0% |
| count decreased | | | | | | | | | | | | |
| TEAE leading to Dose | 0 | 0% | 0 | 0% | 0 | 0% | 0 | 0% | 0 | 0% | 0 | 0% |
| interruption | | | | | | | | | | | | |
| TEAE leading to Dose | 0 | 0% | 0 | 0% | 0 | 0% | 0 | 0% | 0 | 0% | 0 | 0% |
| permanently discontinued | | | | | | | | | | | | |
| TEAE leading to Dose | 0 | 0% | 0 | 0% | 0 | 0% | 0 | 0% | 0 | 0% | 0 | 0% |
| reduced | | | | | | | | | | | | |
| Serious TEAE | 0 | 0% | 0 | 0% | 0 | 0% | 0 | 0% | 1 | 25% | 0 | 0% |

*TEAE: Treatment emergent adverse events

TABLE 3

| | 320 mg QD (N = 3) | | | | 480 mg QD (N = 8) | | | | 600 mg QD (N = 3) | | | |
|---|---|---|---|---|---|---|---|---|---|---|---|---|
| | All grade | | Grade ≥ 3 | | All grade | | Grade ≥ 3 | | All grade | | Grade ≥ 3 | |
| | N | % | N | % | N | % | N | % | N | % | N | % |
| Any TEAE | 3 | 100% | 1 | 33% | 8 | 100% | 4 | 50% | 3 | 100% | 1 | 33% |
| TEAE occurring in ≥10% | | | | | | | | | | | | |
| of all patients | | | | | | | | | | | | |
| Nausea | 1 | 33% | 0 | 0% | 6 | 75% | 0 | 0% | 3 | 100% | 0 | 0% |
| Vomiting | 1 | 33% | 0 | 0% | 4 | 50% | 0 | 0% | 3 | 100% | 0 | 0% |
| Diarrhea | 2 | 67% | 1 | 33% | 4 | 50% | 0 | 0% | 3 | 100% | 0 | 0% |
| Anorexia | 1 | 33% | 0 | 0% | 2 | 25% | 0 | 0% | 1 | 33% | 0 | 0% |
| Constipation | 1 | 33% | 0 | 0% | 0 | 0% | 0 | 0% | 0 | 0% | 0 | 0% |
| Dizziness | 1 | 33% | 0 | 0% | 2 | 25% | 1 | 13% | 0 | 0% | 0 | 0% |
| Dyspepsia | 1 | 33% | 0 | 0% | 0 | 0% | 0 | 0% | 1 | 33% | 0 | 0% |
| Platelet count | 0 | 0% | 0 | 0% | 2 | 25% | 2 | 25% | 1 | 33% | 0 | 0% |
| decreased | | | | | | | | | | | | |
| Neutropenia/Neutrophil | 0 | 0% | 0 | 0% | 2 | 25% | 2 | 25% | 1 | 33% | 1 | 33% |
| count decreased | | | | | | | | | | | | |
| TEAE leading to Dose | 2 | 67% | 1 | 33% | 6 | 75% | 3 | 38% | 2 | 67% | 1 | 33% |
| interruption | | | | | | | | | | | | |
| TEAE leading to Dose | 0 | 0% | 0 | 0% | 1 | 13% | 0 | 0% | 1 | 33% | 1 | 33% |
| permanently discontinued | | | | | | | | | | | | |
| TEAE leading to Dose | 1 | 33% | 0 | 0% | 3 | 38% | 1 | 13% | 1 | 33% | 0 | 0% |
| reduced | | | | | | | | | | | | |
| Serious TEAE | 0 | 0% | 0 | 0% | 4 | 50% | 2 | 25% | 1 | 33% | 1 | 33% |

25 patients have been treated with Compound A in dose-escalation cohorts of a once-daily schedule, with good overall tolerance and a safety profile comparable to other members of the EGFR inhibitor class (data not shown). Treatment-related adverse events of grade ≥3 occurred in patients treated with Compound A 320 mg QD. Gastrointestinal toxicities (diarrhea, nausea, vomiting) were the most common adverse events. Skin toxicities were mild, with no grade ≥3 events reported. Asymptomatic hematologic toxicities of grade ≥3 were observed in high doses (≥480 mg QD).

### 2-3. Case 1: Efficacy in a Patient with EGFR 19Del/T790M/C797S Triple Mutation

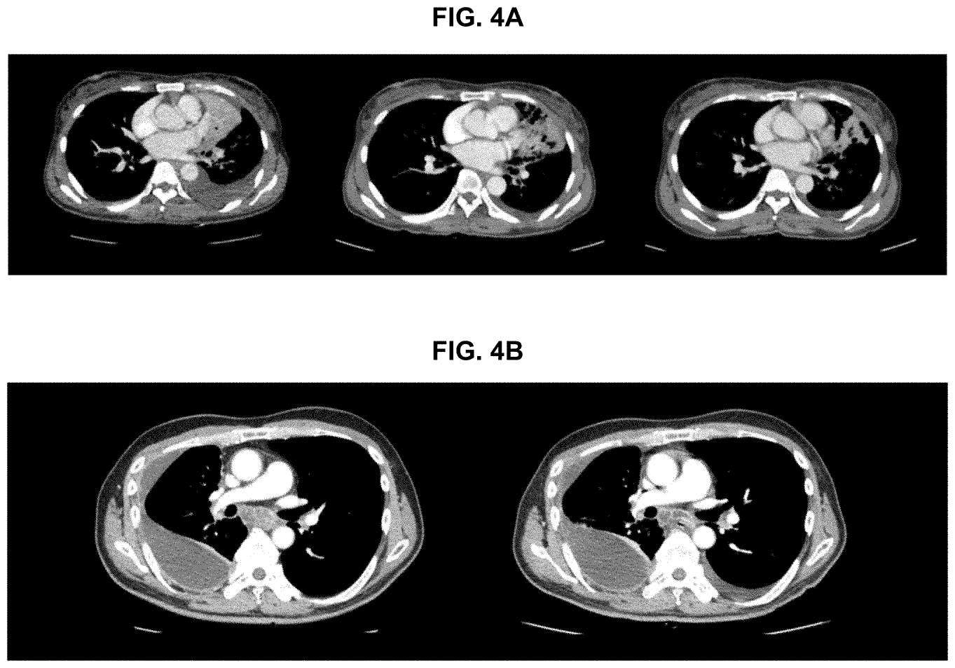

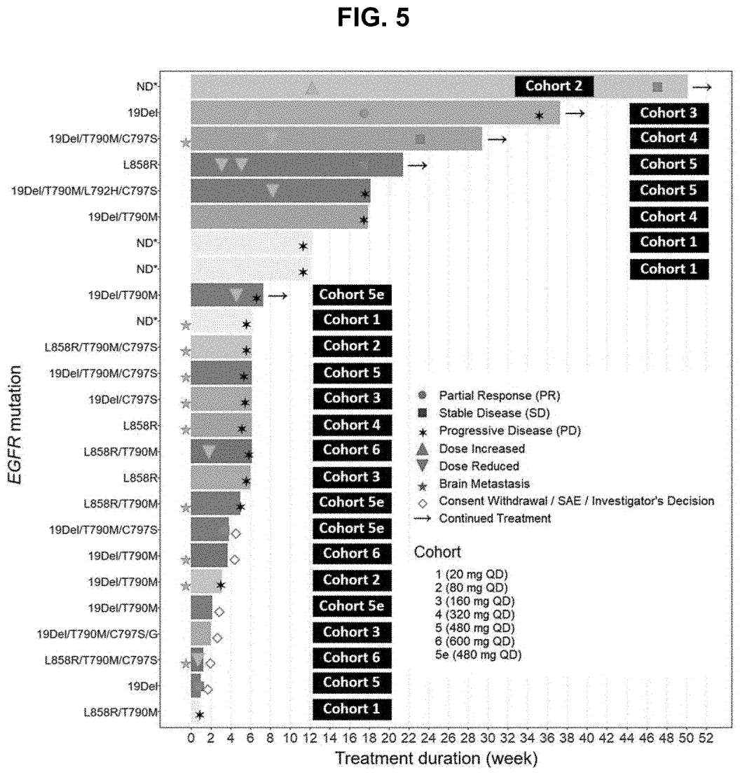

A 52-year-old female patient from Korea had been treated with gefitinib, erlotinib and osimertinib since April 2019 for stage IV lung adenocarcinoma. At diagnosis, EGFR 19Del was found, and subsequently T790M was also detected. She was enrolled in the phase 1 trial (dose-escalation phase) and was allocated to the Compound A 320 mg QD cohort. The dose was reduced to 160 mg QD because of an erythematous skin rash on her forearms. Analysis of ctDNA reported the existence of a triple-mutant clone of EGFR E746_A750del/T790M/C797S. Unexpectedly, JAK2 V617F was also found in blood, which was attributable to the coincidental hematologic disease of myeloproliferative neoplasm. Platelet count was over 500,000/mm3 in this patient prior to the administration of Compound A, therefore, this condition was judged to be concomitant unrelated disease, rather than a Compound A-related adverse event. Considering the potential gains and risks of treatment, the investigator opted to continue, and after 6 weeks of treatment, a computed tomography (CT) scan confirmed considerable improvement in non-target lesions (left pleural effusion and consolidation in the left lung upper lobe; FIG. 4A), while ctDNA analysis found allelic frequency values were stable. A CT scan at week 12 showed further improvements, the investigator response evaluation was stable disease, and the independent central radiology reviewers confirmed partial response since the first follow-up (target lesion shrinkage in the left lung mediastinal lymph node) by Response Evaluation Criteria in Solid Tumors (RECIST) v.1.1 standards.

### 2-4. Case 2: Efficacy in a Patient with EGFR 19Del/T790M/L792H and EGFR 19Del/T790M/C797S Triple Mutations

A 53-year-old male patient from Korea, had been treated with afatinib, osimertinib and chemotherapy, for stage IV lung adenocarcinoma since August 2018. At diagnosis, both EGFR 19Del and T790M were detected. He was enrolled in the phase 1 trial and was allocated to Compound A 480 mg QD. Analysis of ctDNA reported the existence of EGFR variant alleles of E746_A750del, T790M, L792H, and C797S, and allelic context analysis revealed that both L792H and C797S were linked mutually exclusively to T790M. These data show that two clones of triple mutants—EGFR E746_A750del/T790M/L792H and EGFR E746_A750del/T790M/C797S—co-existed in the blood of this patient. After 6 weeks of treatment, CT scan showed shrinkage of the target lesion (subcarinal lymph node conglomerates, 26.3% reduction by the short axis diameter, as assessed by the independent central radiology reviewers) with central density reduction; another upper mediastinal lymph node lesion showed similar changes (FIG. 4B). In this case, molecular changes were more prominent than radiologic tumor shrinkage. We also conducted an enzyme-based assay for L792H-containing triple mutant EGFR (19Del/T790M/L792H), which showed an IC₅₀ value of 13.1 nM.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

### List of References

1. Leonetti A, Sharma S, Minari R, Perego P, Giovannetti E, Tiseo M. Resistance mechanisms to osimertinib in EGFR-mutated non-small cell lung cancer. Br J Cancer 2019;121:725-37.
2. Ramalingam S S, Cheng Y, Zhou C, Ohe Y, Imamura F, Cho B C, et al. Abstract LBA50: Mechanisms of acquired resistance to first-line osimertinib: Preliminary data from the phase III FLAURA study. Ann Oncol Elsevier; 2018;29(Suppl 8):viii740.
3. Papadimitrakopoulou V A, Wu Y-L, Han J-Y, Ahn M-J, Ramalingam S S, John T, et al. LBA51—Analysis of resistance mechanisms to osimertinib in patients with EGFR T790M advanced NSCLC from the AURAS study. Ann Oncol 2018;29:viii741.
4. Lategahn J, Keul M, Klövekorn P, Tumbrink H L, Niggenaber J, Müller M P, et al. Inhibition of osimertinib-resistant epidermal growth factor receptor EGFR-T790M/C797S. Chem Sci 2019;10:10789-801.
5. Bauml J, Cho B C, Park K, Lee K H, CHO E K, Kim D-W, et al. Amivantamab in combination with lazertinib for the treatment of osimertinib-relapsed, chemotherapy-naïve EGFR mutant (EGFRm) non-small cell lung cancer (NSCLC) and potential biomarkers for response. J Clin Oncol Wolters Kluwer; 2021;39 (Suppl 15):9006-9006.
6. Lim S M, Park C W, Zhang Z, Woessner R, Dineen T, Stevison F, et al. Abstract 1467: BLU-945, a fourth-generation, potent and highly selective epidermal growth factor receptor (EGFR) tyrosine kinase inhibitor (TKI) with intracranial activity, demonstrates robust in vivo antitumor activity in models of osimertinib-resistant non-small cell lung cancer (NSCLC). Cancer Res 2021;81(Suppl 13):1467-1467.
7. Mak, A J, Quinn, K J, Espenschied C, Chang K, Chuang H Y, Helman E, et al. Comparison of molecular response calculations for prediction of patient outcome. Proceedings of the 112th Annual Meeting of the American Association for Cancer Research; 2021 April 10-15. Philadelphia, PA, USA. Abstract 401.

What is claimed is:

1. A method for treating or ameliorating symptoms of a cancer with a mutation in a subject in need thereof, comprising administering an effective amount of N-(2-((5-chloro-2-((2-methoxy-4-(4-(4-methylpiperazin-1-yl)piperidin-1-yl)phenyl)amino)pyrimidin-4-yl)amino)phenyl)methanesulfonamide, a prodrug thereof, a hydrate thereof, a solvate thereof, an isomer thereof, an isotope labeled compound thereof, or a pharmaceutically acceptable salt thereof to the subject, wherein the mutation is selected from the group consisting of D(del19), T(T790M), C(C797S), L(L858R), DT, DC, TC, TL, LC, DTC, LTC, and a combination thereof.

2. The method of claim 1, wherein the effective amount is a fixed dose of about 20 mg to about 600 mg.

3. The method of claim 2, wherein the fixed dose is about 20 mg to about 120 mg, about 120 mg to about 200 mg, about 200 mg to about 300 mg, about 300 mg to about 500 mg, or about 500 mg to 600 mg, per day.

4. The method of claim 3, wherein the fixed dose is about 300 mg, about 310 mg, about 320 mg, about 330 mg, about 350 mg, about 360 mg, about 370 mg, about 380 mg, about 390 mg, about 400 mg, about 410 mg, about 420 mg, about 430 mg, about 450 mg, about 460 mg, about 470 mg, about 480 mg, about 490 mg, or about 500 mg.

5. The method of claim 1, wherein the subject is a mammal.

6. The method of claim 2, wherein the administering is oral administration.

7. The method of claim 2, wherein the administering is daily administration.

8. The method of claim 1, wherein the cancer is lung cancer.

9. The method of claim 1, wherein the cancer is non-small cell lung cancer.

10. The method of claim 1, further comprising administering an additional anti-cancer agent.

11. The method of claim 10, wherein the additional anti-cancer agent is a cytotoxic agent or a therapeutic antibody.

12. The method of claim 11, wherein the therapeutic agent is cetuximab.

13. The method of claim 1, further comprising a step of determining whether the subject has the mutation, prior to administering N-(2-((5-chloro-2-((2-methoxy-4-(4-(4-methylpiperazin-1-yl)piperidin-1-yl)phenyl)amino)pyrimidin-4-yl)amino)phenyl)methanesulfonamide, a prodrug thereof, a hydrate thereof, a solvate thereof, an isomer thereof, an isotope labeled compound thereof or a pharmaceutically acceptable salt.

14. The method of claim 13, wherein the subject has previously received or undergoes a chemotherapy or cancer immunotherapy.

15. The method of claim 13, further comprising administering an additional anti-cancer agent.

16. The method of claim 14, the additional anti-cancer agent is a cytotoxic agent or a therapeutic antibody.

17. The method of claim 16, wherein the therapeutic agent is cetuximab, panitumumab, nimotuzumab, or necitumumab.
